(19) United States
(12) Patent Application Publication
(10) Pub. No.: US 2009/0181347 A1
(43) Pub. Date: Jul. 16, 2009

(54) BONE AUGMENTATION PRODUCT FOR GUIDING BONE TISSUE REGENERATION

(76) Inventors: Fu-Yi Lin, Taipei (TW); Yen-Ching Huang, Taipei (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 12/014,700

(22) Filed: Jan. 15, 2008

(51) Int. Cl. A61C 5/00; A61B 17/08; A61F 2/02

(52) U.S. Cl. 433/215; 606/151; 623/23.72

(57) ABSTRACT

A bone augmentation product for guiding bone tissue regeneration includes a soft tissue barrier membrane in the form of a soft sheet, and a tissue-regeneration guiding material being integrally molded to one side of the soft tissue barrier membrane. The tissue-regeneration guiding material may be collagen for directly filling in a defective area on a tooth bed or an area on a bone requiring osteogenesis to thereby simplify the procedures and reduce the difficulty in a surgery.

### FIELD OF THE INVENTION

The present invention relates to a bone augmentation product for guiding bone tissue regeneration, and more particularly to a bone augmentation product for guiding bone tissue regeneration that has simple structure and allows convenient application thereof to a bone defect.

### BACKGROUND OF THE INVENTION

The bone structure at any area of a human body is possibly damaged due to lesion or surgery. Particularly, in a dental surgery, such as tooth extraction, the gum bone might become sunken or too thin, preventing the subsequent operation, such as tooth implantation, from being performed. Under this condition, proper bone tissue regeneration procedures must be done to fill a predetermined bone augmentation product for guiding bone tissue regeneration in the defective or damaged area, so that the defective or damaged gum bone may be reinforced through osteogenesis to meet the required strength and condition for subsequent operation.

FIGS. 1 to 3 show a first conventional bone augmentation product for guiding bone tissue regeneration and the manner of applying the product on a defective area on gum bone. The first conventional bone augmentation product for guiding bone tissue regeneration is suitable for use in the regeneration of a large area of bone tissue. In this case, the surrounding tissue 32 located around the defective area 31 on the tooth bed 3 or the area on the bone requiring osteogenesis, such as gum or muscle, is incised, and an osteogenetic material 4, such as particulates prepared from autogenous bone, synthetic bone, or allogeneic bone, is filled in the defective area or the area requiring osteogenesis or increased thickness on the bone. Thereafter, a tissue barrier membrane 5 or a tissue barrier membrane with a reinforcing skeleton, such as a piece of titanium net, is disposed on the osteogenetic material 4. Finally, the incised surrounding tissue 32 is sutured to allow the wound to recover gradually.

In the above-described first conventional bone augmentation product for guiding bone tissue regeneration and the manner of applying the product on a defective gum bone area, since the osteogenetic material 4 and the tissue barrier membrane 5 or the reinforced tissue barrier membrane are separated from each other, total three steps are required to apply the bone augmentation product for guiding bone tissue regeneration, that is, filling the osteogenetic material 4, disposing the tissue barrier membrane 5, and suturing the surrounding tissue 32. The three steps are separately performed to cause inconveniences in the surgery; and the osteogenetic material 4 and the tissue barrier membrane 5 are not easily joined together to thereby increase the difficulty in the surgery. Moreover, most tissue barrier membranes 5 with reinforcing skeleton are not compatible with the surrounding tissue 32, that is, the gum or the muscle. Therefore, when the osteogenetic material 4 has combined with the surrounding tooth bed 3 or bone, the surrounding tissue 32 must be incised again to take off the tissue barrier membrane 5 with reinforcing skeleton. In this manner, the patient is subject to more pain and the surgery cost is increased.

FIGS. 4 to 5 show a second conventional bone augmentation product for guiding bone tissue regeneration and the manner of applying the product on a defective bone area. After a tooth is extracted, there would be a deep cavity 301 left on the tooth bed 30. In this case, an osteogenetic material 40, such as bone powder prepared from autogenous bone, synthetic bone, or allogeneic bone, is filled in the deep cavity 301, and a tissue barrier membrane 50 is disposed on the osteogenetic material 40. Since the osteogenetic material 40 is in the form of powder and unable to consolidate into a firm structure, it is subject to compression due to external force from cheeks, lips, tongue, and other teeth after having been filled in the cavity 301 on the tooth bed 30. As a result, the osteogenetic material 40 fails to serve as an effective supporting material and form a required shape in the cavity 301 for the bone regeneration. Complicated procedures are also required to apply the second conventional bone augmentation product for guiding bone tissue regeneration on the defective or damaged area.

It is therefore tried by the inventor to develop an improved bone augmentation product for guiding bone tissue regeneration, so as to eliminate the above-mentioned drawbacks.

### SUMMARY OF THE INVENTION

A primary object of the present invention is to provide a bone augmentation product for guiding bone tissue regeneration that has simple structure to thereby simplify the procedures of and reduce the difficulty in applying the product on a desired position, and accordingly, to upgrade the efficiency of surgery.

Another object of the present invention is to provide a bone augmentation product for guiding bone tissue regeneration that has increased structural strength and may easily form a desired bone profile in the bone tissue regeneration.

To achieve the above and other objects, the bone augmentation product for guiding bone tissue regeneration according to the present invention includes a soft tissue barrier membrane in the form of a soft thin sheet, and a tissue-regeneration guiding material being integrally molded to one side of the soft tissue barrier membrane and allowing free manipulation for filling in a defective area on tooth bed or in a desired area for bone osteogenesis.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIGS. 1 to 3 show a first conventional bone augmentation product for guiding bone tissue regeneration and the manner of applying the product on a defective bone area;

FIGS. 4 to 5 show a second conventional bone augmentation product for guiding bone tissue regeneration and the manner of applying the product on a defective bone area;

FIGS. 6 to 8 show a bone augmentation product for guiding bone tissue regeneration according to a first embodiment of the present invention and the manner of applying the product on a defective bone area; and

FIGS. 9 to 10 show a bone augmentation product for guiding bone tissue regeneration according to a second embodiment of the present invention and the manner of applying the product on a defective bone area.

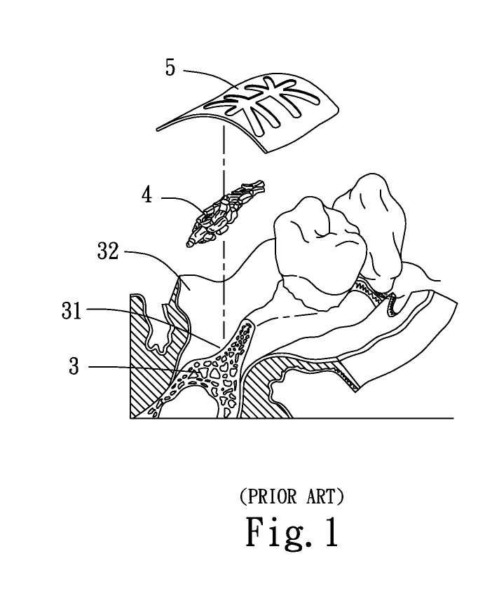

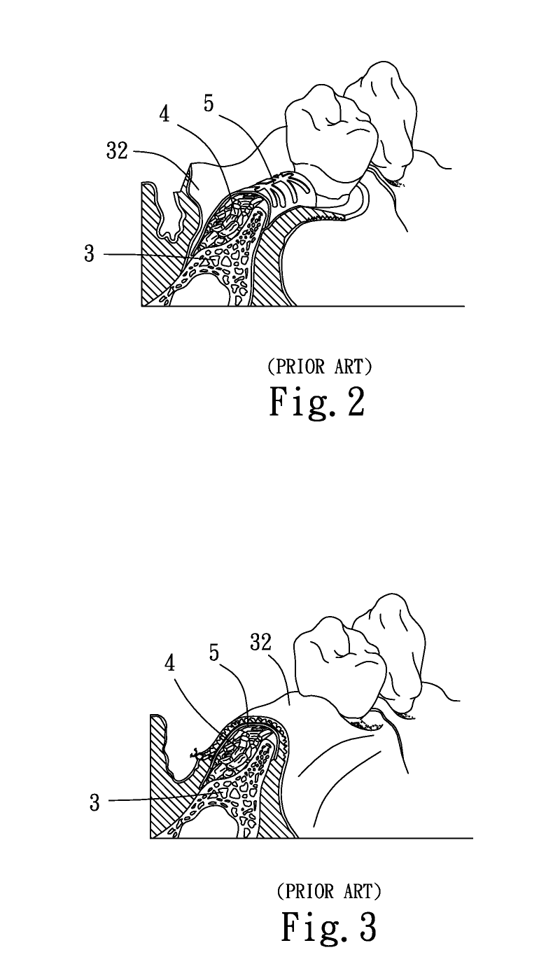

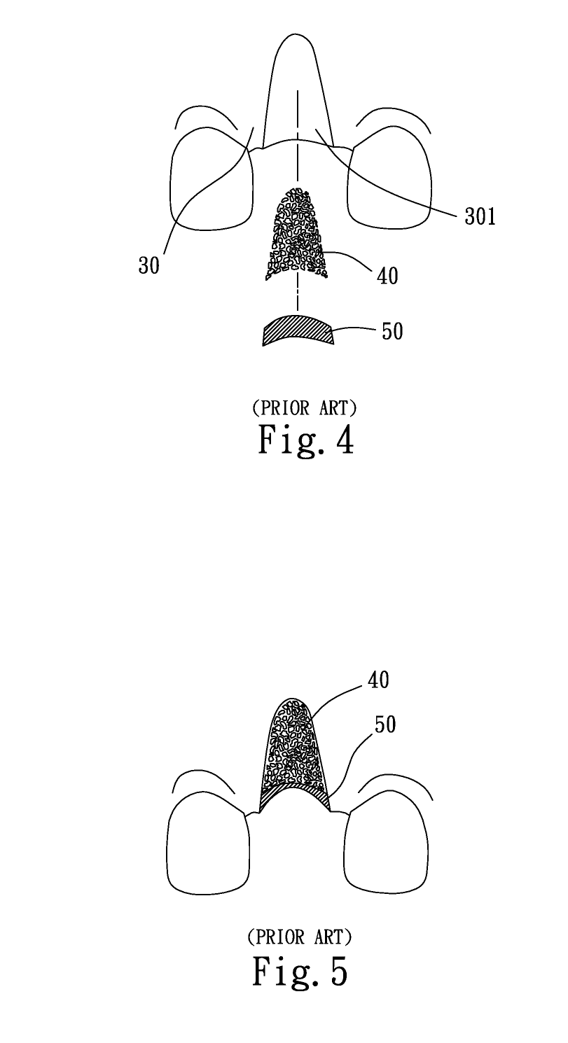

### DETAILED DESCRIPTION OF THE PREFERRED EMBODIMENTS

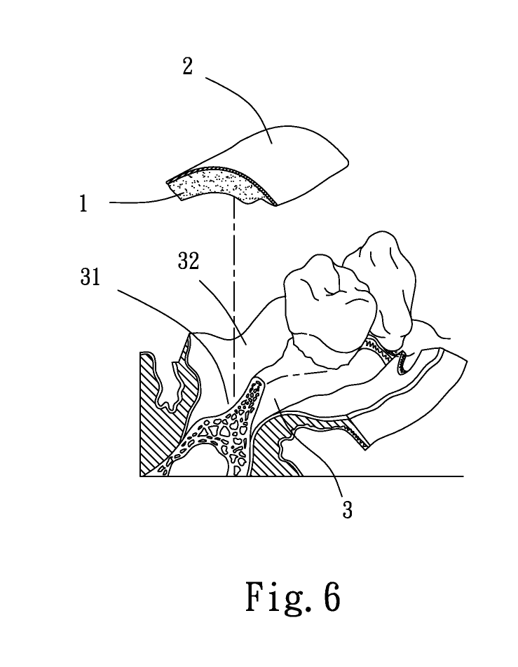

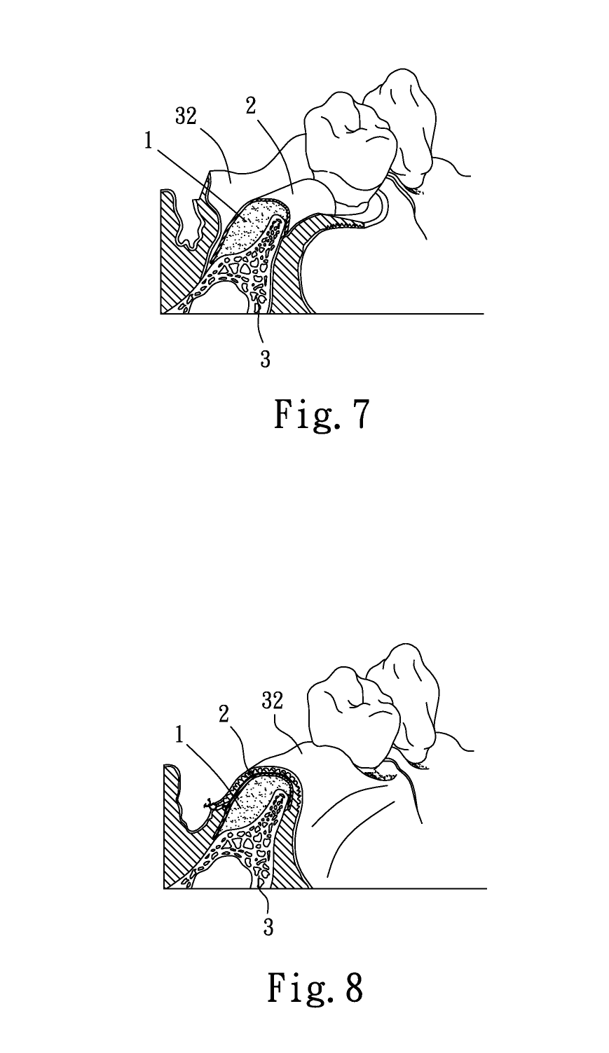

Please refer to FIGS. 6 to 8. A bone augmentation product for guiding bone tissue regeneration according to a first embodiment of the present invention includes a tissue-regeneration guiding material 1 and a soft tissue barrier membrane 2. The tissue-regeneration guiding material 1 may be collagen. The soft tissue barrier membrane 2 is integrally molded to one side of the tissue-regeneration guiding material 1. The tissue-regeneration guiding material 1 may be molded into a predetermined shape as required. The soft tissue barrier membrane 2 may be divided into two types, one of which is human tissue compatible and absorbable, while the other one is not human tissue compatible and absorbable. The soft tissue barrier membrane 2 compatible with and absorbable by human tissue may be collagen membrane, artificial dermis, or allogeneic dermis. The soft tissue barrier membrane 2 bio compatible with and nonabsorbable by human tissue may be polytetrafluorothylene (PTFE) membrane.

To use the present invention, first incise the surrounding tissue 32, such as gum or muscle, that is located around the defective area 31 on the tooth bed 3 or the area requiring osteogenesis on the bone; and dispose the bone augmentation product for guiding bone tissue regeneration on the defective bone area or the bone area requiring osteogenesis or increased thickness. Finally, suture the incised surrounding tissue 32 and allow the wound to recover gradually. No other operation is needed. Therefore, the present invention effectively simplifies the operating procedures and reduces the difficulty in the whole surgery. Meanwhile, with the tissue-regeneration guiding material 1 integrally molded to one side of the soft tissue barrier membrane 2, the bone augmentation product for guiding bone tissue regeneration of the present invention provides increased structural strength, and can therefore support and maintain a profile desired for the bone in the bone tissue regeneration and thereby upgrade the surgical quality.

For the present invention to be more stable in practical application thereof, a biopsy is performed on the regenerated bone to observe its growth at different stages of three weeks, two months, three months, and four months after the surgery. The biopsy is performed on the regenerated bone by using a circular saw having an inner diameter of 2.5 mm and an outer diameter of 3.5 mm to obtain a sample of the regenerated bone. The obtained bone sample is fixed in formalin for more than 48 hours, and is then decalcified, dehydrated, embedded in paraffin, sectioned, de-waxed, dyed by HE (hematoxylin-eosin) dyeing, and mounted or sealed. The sample so treated is observed under microscope at different growth stages, and the observed results are as follows:
1. On the 21^{st} day, about 25% to 30% of new bone is formed. The residual tissue-regeneration guiding material 1 observed under microscope has fibrous tissue mother cells grown between cell-wall-like frames, and there are capillaries formed in immature bone tissue. There is not active osteoclast concentration, and it is able to directly form relatively mature regenerated bone.
2. After two months, about 50% to 60% of new bone is formed. There is still a small amount of residual tissue-regeneration guiding material 1 observed under microscope. However, more amount of new bone has been formed.
3. After three months, about 70% of new bone is formed. Only a very small amount of residual tissue-regeneration guiding material 1 is observed under microscope, and more mature bone is formed.
4. After four months, about 90% of new bone is formed, and all the bone is mature regenerated bone.

From the above observations, it is found the bone augmentation product for guiding bone tissue regeneration according to the present invention can effectively grow to form the desired regenerated bone.

In the case the soft tissue barrier membrane 2 is a material compatible with and absorbable by human tissue, it will be compatible with the patient's body tissue when the surrounding tissue 32 is sutured and the wound is recovered. Therefore, it is not necessary to perform an other surgery to takeoff the soft tissue barrier membrane 2. On the other hand, in the case the soft tissue barrier membrane 2 is a material bio compatible with and nonabsorbable by human tissue, the surrounding tissue 32 (that is, the gum or the muscle) must be incised again when the tissue-regeneration guiding material 1 has combined with the tooth bed 3 or the bone, so as to take off the soft tissue barrier membrane 2.

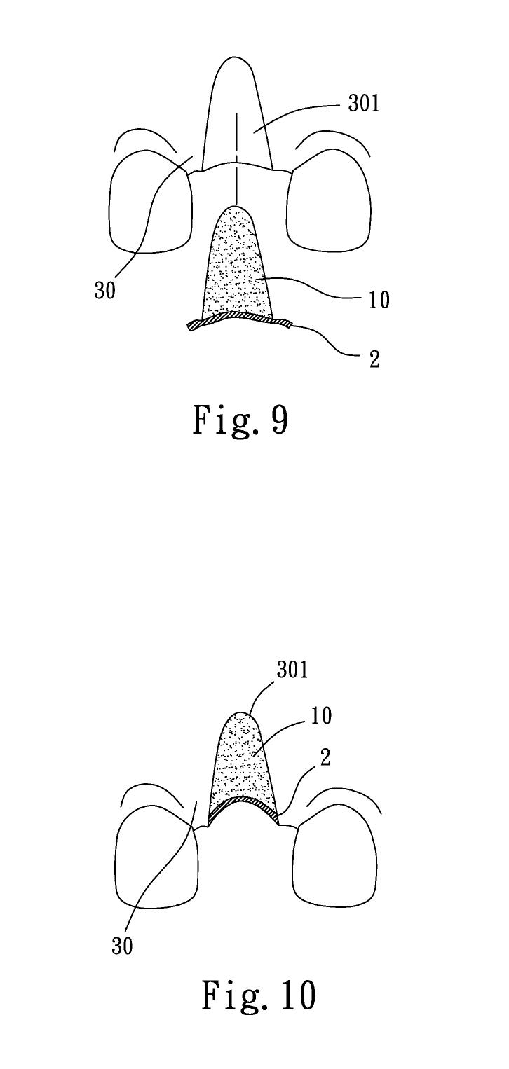

FIGS. 9 to 10 show a bone augmentation product for guiding bone tissue regeneration according to a second embodiment of the present invention and the manner of applying it on the defective bone area. The bone augmentation product for guiding bone tissue regeneration according to the second embodiment of the present invention includes a tissue-regeneration guiding material 10 and a soft tissue barrier membrane 2. The tissue-regeneration guiding material 10 may be collagen and may be molded into a desired shape. The soft tissue barrier membrane 2 is integrally molded to one side of the tissue-regeneration guiding material 10.

When there is a small area on the tooth bed 3 or the bone, such as a deep cavity 301 formed on the tooth bed 3 after tooth extraction, that requires bone tissue regeneration, the tissue-regeneration guiding material 10 may be directly filled in the deep cavity 301 with the soft tissue barrier membrane 2 located at an opening of the cavity 301. Since the tissue-regeneration guiding material 10 may be molded into a desired shape, it is able to support and maintain the cavity 301 in a desired bone profile in the bone regeneration. The second embodiment of the present invention can be easily applied on the defective bone area to provide the same convenience in surgery as the first embodiment.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A bone augmentation product for guiding bone tissue regeneration, comprising:
a soft tissue barrier membrane in the form of a soft sheet; and
a tissue-regeneration guiding material being integrally molded to one side of the soft tissue barrier membrane for filling a defective area on a tooth bed or an area on a bone requiring osteogenesis.

2. The bone augmentation product for guiding bone tissue regeneration as claimed in claim 1, wherein the tissue-regeneration guiding material is collagen.

3. The bone augmentation product for guiding bone tissue regeneration as claimed in claim 1, wherein the soft tissue barrier membrane is selected from the group consisting of collagen membrane, artificial dermis, and allogeneic dermis.

4. The bone augmentation product for guiding bone tissue regeneration as claimed in claim 1, wherein the soft tissue barrier membrane is a polytetrafluorothylene (PTFE) membrane.
